(19) United States
(12) Patent Application Publication
(10) Pub. No.: US 2008/0138829 A1
(43) Pub. Date: Jun. 12, 2008

(54) Method For Detecting and/or Determining the Concentration of at Least One Ligand

(75) Inventors: Mirko Lehmann, Ebmat-Kuppel (CH); Ingo Freund, Freiburg (DE); Holger Klapproth, Freiburg (DE); Sonja Mohry, Freiburg (DE)

Correspondence Address: THE WEBB LAW FIRM, P.C., 700 KOPPERS BUILDING, 436 SEVENTH AVENUE, PITTSBURGH, PA, 15219, US

(73) Assignees: Micronas GmbH, Freiburg i.Br. (DE); Micronas Holding GmbH, Freiburg i.Br. (DE)

(21) Appl. No.: 11/665,169

(22) PCT Filed: Oct. 12, 2005
(86) PCT No.: PCT/EP05/10976

§ 371 Date: Oct. 8, 2007

(30) Foreign Application Priority Data
Oct. 13, 2004 (DE) 10 2004 050 32.0

(51) Int. Cl. G01N 21/76; G01N 33/543; G01N 33/53

(52) U.S. Cl. 435/7.1; 436/518; 422/52

(57) ABSTRACT

The invention relates to a method for detecting and/or determining the concentration of a ligand contained in a solution to be analyzed, during which a receptor, which can enter into a specific bond with the ligand, is immobilized on a semiconductor chip at a test location. In order to bind the ligand to the receptor, the solution is applied to the test location. A luminescence radiation or a color change is generated according to the binding of the ligand to the receptor. A radiation measurement signal is picked up with the aid of a radiation receiver integrated in the semiconductor chip, whereas the solution and/or an auxiliary liquid that does not contain the luminophore is in contact with the radiation receiver. While the generation of luminescence radiation is prevented and the radiation receiver is in contact with the auxiliary liquid and/or with a substitute liquid, a darkness measurement signal is measured for the radiation receiver. The radiation measurement signal is compensated for with the darkness measurement signal

The invention relates to a method for detecting and/or determining the concentration of at least one ligand, which is assumed to be contained in a solution to be analyzed, wherein at least one receptor, which enters into a specific bond with the ligand when the receptor contacts the ligand, is immobilized on a semiconductor chip on at least one test location, wherein the solution for binding the ligand to the receptor is applied to the test location, wherein the solution is removed from the test location and an auxiliary liquid containing a luminophore that is stimulated to emit a luminescence radiation according to the bond of the ligand to the receptor is applied to the test location, and wherein a radiation measurement signal is picked up with the aid of at least one radiation receiver that is sensitive to the luminescence radiation and integrated in the semiconductor chip.

In addition, the invention relates to a method for detecting and/or determining the concentration of at least one ligand that is assumed to be contained in a solution for analysis, wherein at least one receptor, which enters into a specific bond with the ligand when the receptor contacts the ligand, is immobilized on a semiconductor chip on at least one test location, wherein the solution for binding the ligand to the receptor is applied to the test location, wherein at least one luminophore is immobilized on the test location on the receptor according to the bond of the ligand to the receptor, wherein the test location is irradiated with an excitation radiation that contains at least one wavelength at which the luminophore is stimulated to emit a luminescence radiation, and wherein a radiation measurement signal is picked up with the aid of at least one radiation receiver that is sensitive to the luminescence radiation and that is integrated in the semiconductor chip.

Furthermore, the invention relates to a method for detecting and/or determining the concentration of at least one ligand that is assumed to be contained in a solution for analysis, wherein at least one receptor, which enters into a specific bond with the ligand when the receptor contacts the ligand, is immobilized on a carrier on at least one test location, wherein the solution for binding the ligand to the receptor is applied to the test location, wherein a reagent is brought into contact with the test location and is converted according to the bond of the ligand to the receptor into an indicator that differs in color from the reagent, and wherein the test location is irradiated with an optical radiation.

In a method disclosed in DE 102 51 757 A1 for determining the concentration of a ligand contained in a solution, receptors are immobilized on the surface of a semiconductor chip on test locations, on which in each case an optical radiation receiver and a surface occupancy sensor are integrated in the semiconductor chip. To determine the concentrations of the ligands, the solution is applied to the test locations so that it comes into contact with the receptors. The receptors have epitopes that enter into specific bonds with the ligands upon contact therewith. The semiconductor chip is washed to remove components of the solution that are not bound to a receptor. An auxiliary solution containing a luminophore that is stimulated to emit a luminescence radiation according to the bond of the ligand to the receptor is then applied to the test location. Furthermore, the area of the surface of the semiconductor chip occupied with the receptors in each case is measured on the test locations with the aid of the area occupancy sensors. The concentration of the ligand on the individual test locations is determined according to the law of mass action with the aid of the measurement signals from the radiation receivers and the area occupancy sensors as well as a bond constant. This method has proven effective in practice, especially as it permits a direct measurement of the concentration of the ligands over a wide range of concentrations. The fact that the measurement results in solutions that contain the ligands only in low concentrations are relatively inaccurate is a disadvantage of the method.

In addition, a method for determining the concentration of a ligand contained in a solution is disclosed in DE 102 51 757 A1, in which the bond of the ligand to the receptors is detected with the aid of a luminophore, which is indirectly bound to a receptor-ligand complex via a detection antibody. The semiconductor chip is washed to remove free luminophores not bound to a receptor. Afterwards, the luminophores still bound to the receptors via the detection antibodies are irradiated with an excitation radiation. The spectrum of the excitation radiation has a stimulation wavelength at which the luminophore is stimulated to emit the luminescence radiation, which is detected with the aid of the radiation receivers. Even in this method, however, the measurement accuracy could still be improved.

It is therefore the object of the invention to develop a method of the aforementioned type and a device that permit a high measurement or detection accuracy, especially with low ligand concentrations.

With regard to the aforementioned method in which a chemical luminescence radiation is generated according to the bond of the ligand to the receptor, this object is achieved in that the radiation measurement signal is picked up while the auxiliary liquid is in contact with the radiation receiver, in that the radiation receiver is brought into contact with a substitute liquid differing from the auxiliary liquid in which no luminescence radiation is generated, in that while the radiation receiver is in contact with a substitute liquid, a darkness measurement signal is measured for the radiation receiver, and the radiation measurement signal is compensated for with the darkness measurement signal. The object is achieved with regard to the device with the characteristics of claim 16.

An auxiliary liquid is to be understood as a liquid in which the measurement of the luminescence radiation and/or of a color change occurring according to the bond of the ligand to the receptor takes place. A substitute liquid is to be understood as a liquid that—when it is in contact with the at least one radiation receiver—has more or less the same electrical influence on the measurement signal of the radiation receiver as the auxiliary liquid. No luminescence radiation or color change occurs in the substitute liquid. A specific bond is to be understood as a covalent bond and/or a chemical bond.

Surprisingly, it turned out that the auxiliary liquid remaining in contact with the radiation receivers during the measurement of the radiation measurement signal influences the darkness current or the darkness voltage of the radiation receiver, whereby measurement imprecisions can be generated, particularly for low radiation measurement signal levels, if the darkness current or the darkness voltage is not taken into consideration. In the solution of the invention, these measurement imprecisions are avoided in that a darkness measurement signal is picked up and the radiation measurement signal is compensated for with said darkness measurement signal while the semiconductor chip coated with the receptor-ligand complex(es) formed by the bond of the ligand to the receptor is in contact with the auxiliary liquid and/or a corresponding substitute liquid. In an advantageous manner, the method of the invention permits a high measurement or detection precision even with low ligand concentrations. The at least one ligand and the at least one receptor can be biocomponents or biomolecules, examples of which include nucleic acids or derivatives thereof (DNA, RNA, PNA, LNA, oligonucleotides, plasmids, chromosomes), peptides, proteins (enzymes, proteins, oligopeptides, cellular receptor proteins and complexes thereof, peptide hormones, antibodies and fragments thereof), carbohydrates and derivatives thereof, especially glycosylated proteins and glycosides, fats, fatty acids, and/or lipids. Furthermore, the receptor and/or the ligand can be biological cells (microorganisms and/or cells of higher organisms) and/or subcomponents thereof, such as cellular organelles. The receptor and/or the ligand can also be chemical substances.

The substitute liquid can be found experimentally by means of a comparison test, in which the darkness measurement signal for various fluids is picked up and compared with the radiation measurement signal that is measured in the auxiliary fluid when no ligand is bound to the receptor(s). The liquid in which the darkness measurement signal has the greatest coincidence with the respective radiation measurement signal of the auxiliary liquid is used as the substitute liquid. The liquids chosen for the comparison test must fulfill the following conditions:
1. The specific bonds in the liquid are sufficiently stable,
2. The enzyme and/or the luminophore are not damaged by the liquid,
3. No chemiluminescence radiation capable of being detected by the radiation receiver is generated in the liquid upon contact with the receptor-ligand complexes.

In a preferred exemplary embodiment of the invention, the at least one ligand is directly marked and/or marked with at least one enzyme via at least one detection antibody bound to the ligand (bonding assay), wherein the luminophore is selected so that, in the presence of the enzyme, it can be stimulated directly or indirectly to emit luminescence radiation by the action of a chemical substance contained in the auxiliary liquid. In direct marking, the enzyme is directly bound to the ligand. In indirect marking, the enzyme is bound to the ligand via a bridge of at least two specific bonds. To this end, a first specific bond can be provided, for example, between the ligand and a biotin-marked detection antibody and a second specific bond can be provided between the biotin and a streptavidin molecule, which is bound to the enzyme. Examples of specific bonds include streptavidin-biotin bonds, digoxigenin-antidigoxigenin bonds, and sense and antisense DNA bonds.

It is advantageous if the at least one enzyme is a peroxidase enzyme, preferably horseradish peroxidase, and if the luminophore is luminol and the chemical substance is hydrogen peroxide. Experiments have shown that an especially high detection sensitivity is attained with this combination. Other enzymes and luminophores, however, can also be used with the method of the invention, for example, luciferase and luciferin and/or alkaline phosphatases and CDP-Star® or Disodium 2-chloro-5-(4-methoxyspiro{1,2-dioxetane-3,2(5-chloro)-tricyclo[3.3.1.13,7]decan}-4-yl)-1-phenyl phosphate.

It is advantageous if the auxiliary liquid contains hydrogen peroxide and luminol and the substitute liquid preferably contains a phosphate buffered saline solution (PBS). Such an auxiliary liquid is commercially available under the trade name ECL®. The corresponding solutions are available at low cost and permit a high measurement or detection precision.

The method of the invention is also suitable for competitive assay systems. With such systems, at least one competitor marked with the enzyme is added to the solution to be analyzed, and then the solution is applied to the test location.

The present object is achieved with the aforementioned method in which the luminophore is stimulated to emit a luminescence radiation with the aid of the excitation radiation in that the radiation measurement signal is picked up while the solution and/or an auxiliary liquid that does not contain the luminophore is in contact with the radiation receiver, in that while the generation of the luminescence radiation is prevented, a darkness measurement signal is measured for the radiation receiver, in that the radiation receiver remains in contact with the solution, the auxiliary liquid, and/or a substitute liquid while the darkness measurement signal is being picked up, and in that the radiation measurement signal is compensated for by the darkness measurement signal. With regard to the device, the present object is also achieved with the characteristics of claim 17.

With the method, the solution and/or the substitute liquid is chosen so that it/they has/have about the same electrical influence on the measurement signal of the radiation receiver in the event that the radiation receiver comes in contact with the solution and/or a substitute liquid different from the solution and the auxiliary liquid.

In this solution as well, the radiation measurement signal as well as the darkness measurement signal are thus measured with a “wet” radiation receiver. The generation of the luminescence radiation is suppressed during the darkness measurement by switching off the excitation radiation and/or replacing the auxiliary liquid with a substitute liquid that does not contain the luminophore. In contrast to a method in which the luminescence radiation is measured with a dry semiconductor chip, the measurement of the radiation measurement signal in the substitute liquid permits a higher quantum yield of the luminophore. Furthermore, artifacts such as optical reflections on the surface of the semiconductor chip, which reduce the precision of the measurement, are avoided as much as possible.

The method of the invention can be used with the on-line polymerase chain reaction (PCR). With the aid of the PCR, it is possible to reproduce a specific section between two known sequences from a small quantity of DNA. To this end, two oligonucleotide primers that bind in opposite directions to the complementary strand of a known DNA sequence are needed. In addition, the nucleotides dATP, dCTP, dGTP, and dTTP, as well as a Taq polymerase that is capable of extending the DNA sequence out from the primers, must be added along with the sequence to be amplified. In the on-line PCR, the luminophore is contained in the analyzing solution or is added thereto. The solution containing the free luminophores is in contact with the radiation receiver during the measurement of the luminescence radiation. This contact can also be established via a thin wave conducting layer, which is arranged on the radiation receiver and preferably monolithically integrated therewith. During the picking up of the darkness measurement signal, the radiation receiver can also be in indirect contact with the solution, the auxiliary liquid, and/or the substitute liquid via the wave conducting layer.

After bringing the solution in contact with the test location, it is obviously also possible to remove any free luminophores still present thereon from the test location and apply an auxiliary liquid that does not contain the luminophore thereto after the solution is brought into contact therewith. The luminescence radiation is then measured while the auxiliary fluid remains in contact with the radiation receiver. The luminophores can be applied to the test location together with the solution to be analyzed. It is also conceivable, however, to apply the solution without the luminophores to the test location, and then to remove the solution from the test location, for example, by washing and afterwards bring the luminophores into contact with the test location.

In an embodiment of the invention, the at least one ligand is directly marked with a luminophore and/or marked with a luminophore via at least one detection antibody bound to the ligand (bond assay), wherein during the picking up of the radiation measurement signal, the luminophore is irradiated with an excitation radiation having a wavelength different from that of the luminescence radiation and to which the radiation detector is insensitive, in order to generate said luminescence radiation. As a luminophore, preference is given to an upward-converting luminophore, in which the wavelength of the luminescence radiation is smaller than the excitation wavelength. Such upward-converting luminophores are known per se, for example, from EP 0 723 146 A1.

The method is also suitable for competitive assays. To this end, at least one competitor marked with the luminophore is added to the solution to be analyzed, and the solution is subsequently applied to the test location.

The present object is also achieved by a method for detecting and/or determining the concentration of at least one ligand that is assumed to be contained in the solution to be analyzed,
(a) wherein at least one receptor capable of entering into a specific bond with the ligand is immobilized on at least one test location on a semiconductor chip when said receptor contacts said ligand,
(b) wherein the solution for binding the ligand to the receptor is applied to the test location,
(c) wherein a reagent is brought into contact with the test location and is converted by a chemical reaction, according to the bond of the ligand, to an indicator that is different in color from the reagent,
(d) wherein the test location is irradiated with an optical radiation,
(e) wherein a radiation measurement signal is picked up with the aid of at least one radiation receiver that is integrated in the semiconductor chip and sensitive to the color change occurring in the chemical reaction,
(f) while the solution and/or an auxiliary liquid containing the reagent are/is in contact with the radiation receiver,
(g) wherein a darkness measurement signal for the radiation receiver is measured while the generation of the optical radiation is being prevented and/or the indicator is being maintained remote from the test location,
(h) wherein the radiation receiver is in contact with the solution or the auxiliary liquid and/or the substitute liquid as the darkness measurement signal is being picked up,
(i) and wherein the radiation measurement signal is compensated for with the darkness measurement signal.

### The present object is also achieved with regard to the device with the characteristics of claim 18.

An auxiliary liquid is to be understood as a liquid differing from the solution and not containing the reagent. In claim 9, a substitute liquid is to be understood as a liquid differing from the solution and the auxiliary liquid that, when it is in contact with the at least one radiation receiver, has about the same electrical influence on the measurement signal of the radiation receiver as the liquid that is contact with the radiation receiver while the radiation measurement signal is being picked up.

In this solution also, the radiation measurement signal as well as the darkness measurement signal are thus measured while the radiation receiver is in contact with a liquid. The specific bond is detected with the aid of the color change reaction. The indicator can contain, for example, methyl red and/or bromthymol blue, the color of which can change according to the pH from orange to yellow to turquoise. The ligand can contain a urobilinogen, bilirubin, a ketone, ascorbic acid, glucose, a protein, blood, pH, nitrite, and/or leukocytes. The enzymes glucose oxidase and peroxidase can be immobilized on the test location to detect glucose. Glucose detection can take place via a coupled enzyme reaction. To this end, the glucose is oxidized to gluconic acid by glucose oxidase, and hydrogen peroxide is generated. The peroxidase oxidizes a redox indicator, which turns from green to yellow, with the hydrogen peroxide thus generated.

In a preferred embodiment of the invention, the semiconductor chip has a plurality of radiation receivers, wherein a radiation measurement signal and a darkness measurement signal are preferably always picked up for each of these radiation receivers, and for each radiation receiver, the radiation measurement signal allocated thereto is always compensated for with the darkness measurement signal allocated thereto. To this end, receptors can be immobilized either on all of the radiation receivers or only on some of the radiation receivers. A blind signal can be picked up with the aid of at least one radiation receiver on which no receptor is immobilized. The blind measurement signal can be subtracted from the measurement signal of at least one radiation receiver having at least one receiver in order to compensate for background radiation and/or a background color change not generated according to the bond of a ligand to a receptor.

In an especially advantageous embodiment of the invention, at least one receptor is always immobilized on at least two test locations, wherein a radiation measurement signal and a darkness measurement signal are always picked up for the radiation receivers allocated to said test locations, and wherein the individual radiation measurement signals are always compensated for with the darkness measurement signal allocated to them. In experiments it has been shown that the measurement results show relatively wide scatterings when only a single darkness measurement is carried out for all radiation receivers. These scatterings can be reduced when an individual darkness signal measurement is performed in each case for each single radiation receiver and the radiation measurement signals are then compensated for in each case with the darkness signals allocated to them, for example, by subtracting the darkness measurement signal from the radiation measurement signal. The method then permits an even greater measurement precision. The darkness measurement signals can be picked up for a plurality of test locations simultaneously. Obviously, the darkness signals can also be measured after each other for individual test locations or for groups that in each case comprise a plurality of test locations.

It is advantageous if the same receptors are immobilized on the semiconductor chip on at least two test locations on which the solution to be analyzed is applied, if at least one radiation measurement signal is always picked up and compensated for with a darkness measurement signal for said test locations, and if an average value signal is formed from the compensated measurement signals thus detected. By this means, it is also possible to compensate for additional scatterings caused by differences in the radiation receivers and/or differences in the measurement pickup. By this means, the method permits an even greater measurement or detection precision. All of the test locations located on the semiconductor chip can be included in the formation of the average value. It is also conceivable, however, to generate the average value signal with regard to only a fraction of the test locations located on the semiconductor chip. To this end, it is even possible to provide a plurality of groups of test locations on the semiconductor chip in which the same receptors are always arranged on test locations that belong to the same group and different receptors are arranged on test locations that belong to different groups. It is possible to detect a plurality of ligands and/or determine their concentration in the solution with just one semiconductor chip by this means.

In a preferred embodiment of the invention, the radiation receiver is a photodiode that is connected to an electrical power supply unit for charging its barrier-layer capacity in a reverse biasing, wherein afterwards the connection to the electrical power supply is interrupted, and wherein the electrical voltage is picked up as a radiation measurement signal and/or a darkness measurement signal on the photodiode during the interruption. By precharging the photodiode in reverse biasing, a low barrier-layer capacity is established on the photodiode during the measurement of the radiation or the darkness measurement signal. A wide bandwidth and a high measurement sensitivity are permitted by this means.

In a practical embodiment of the method, the darkness measurement signal is approximated to a line through an intercept, wherein the slope of the line is determined and wherein the radiation measurement signal is compensated for with reference to the slope. The at least one radiation measurement signal can then easily be compensated for, e.g., with the aid of an evaluator integrated in the semiconductor chip.

A wash solution is practically used as a substitute liquid, wherein the at least one test location is rinsed with the wash solution after the application of the solution to be analyzed, wherein the darkness measurement signal is picked up afterwards, and wherein the wash solution is then replaced with the auxiliary solution and the radiation signal is picked up. The darkness measurement signal is therefore measured right after the washing of the test location while the wash solution is still on the test location. The wash solution thus fulfills a double function, and in addition to washing the semiconductor chip, it also serves as a substitute liquid for measuring the darkness measurement signal. Because the application of an additional substitute liquid is dispensed with, the total time required for the measurement is shortened, which is especially advantageous with unstable ligands. Obviously, a reverse procedure is also conceivable, in which, for example, the darkness measurement signal is first picked up in the substitute solution and the radiation signal is picked up afterwards in the auxiliary solution.

Embodiments of the invention are explained in greater detail with reference to the drawing, in which:

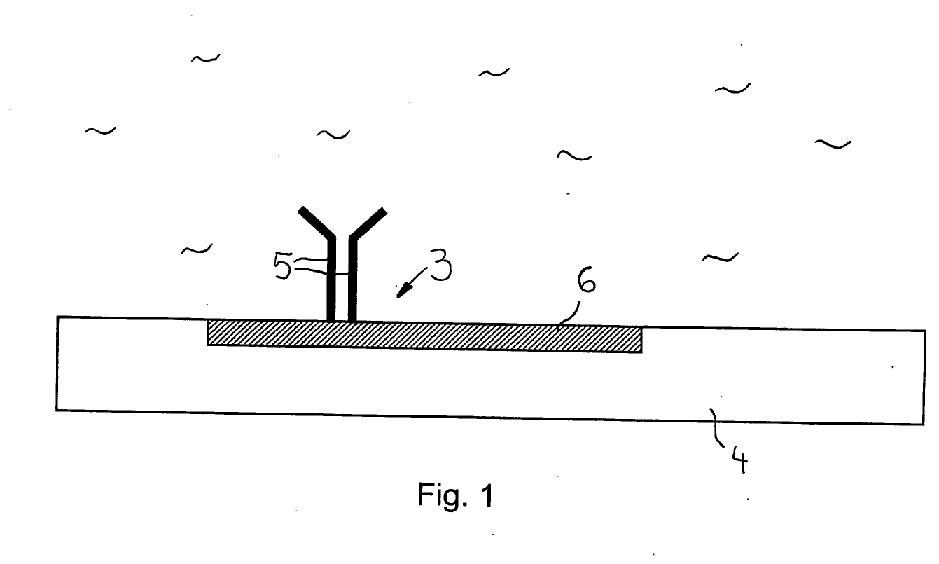

FIG. 1 shows a semiconductor chip on which a receptor is immobilized,

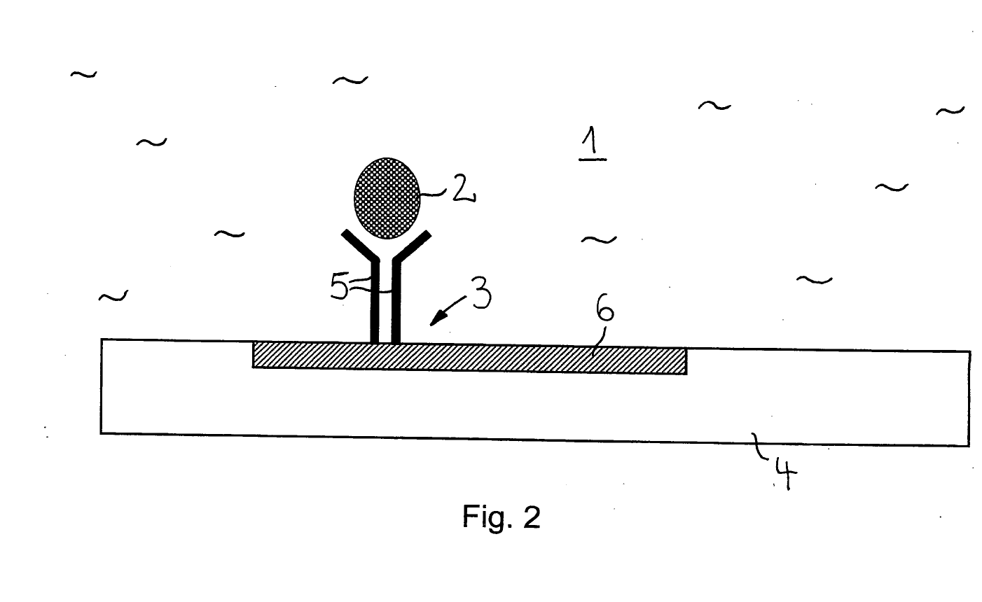

FIG. 2 shows a representation similar to FIG. 1 wherein, however, a ligand is bound to the receptor,

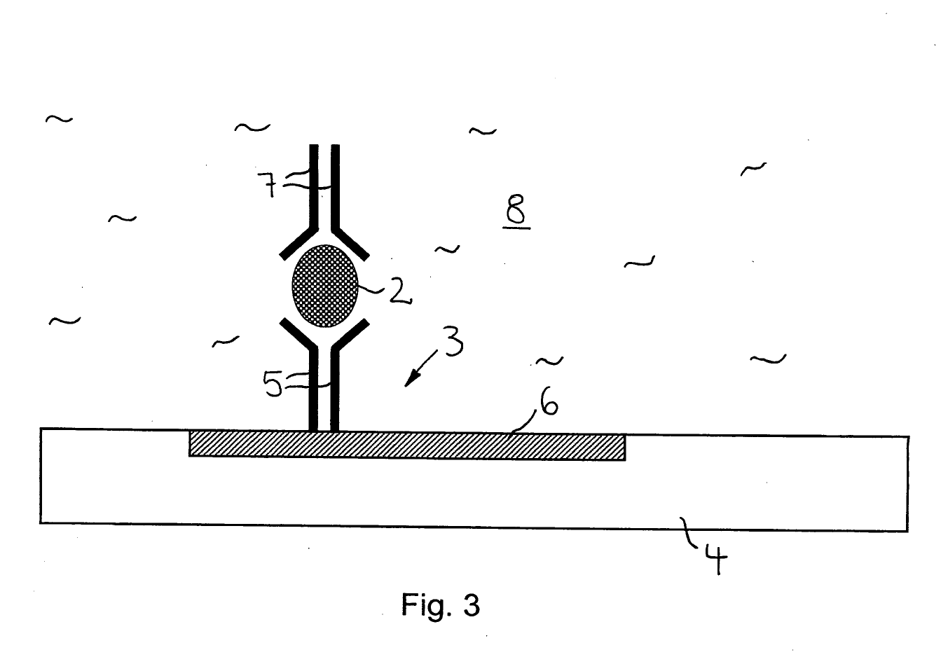

FIG. 3 shows a representation similar to FIG. 2 wherein, however, a detection antibody is bound to the ligand,

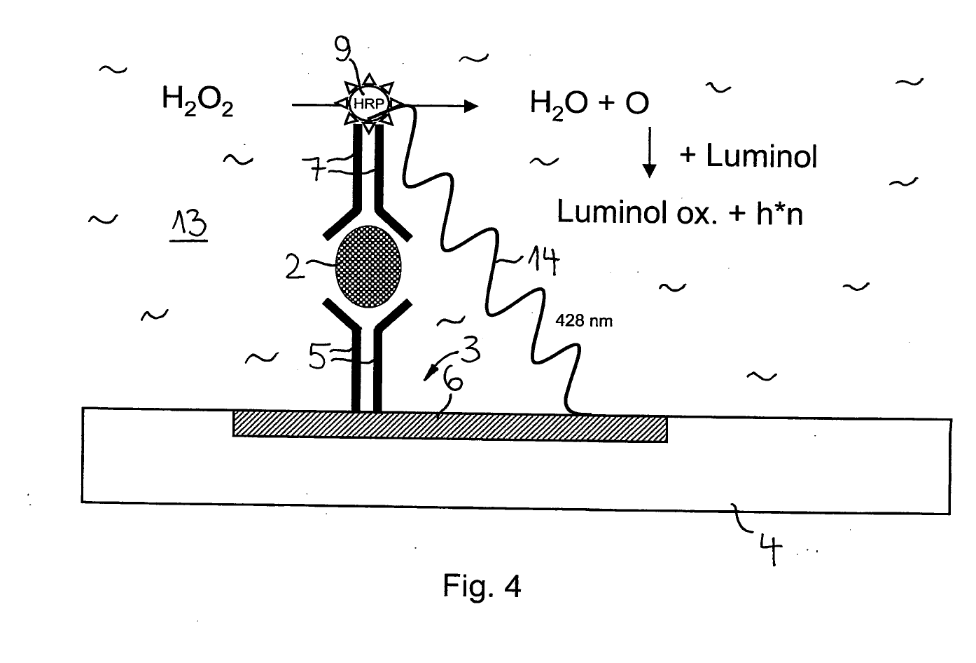

FIG. 4 shows a schematic representation of a sandwich ELISA assay (Enzyme-Linked Immunosorbent Assay), in which a chemical luminescence radiation is generated,

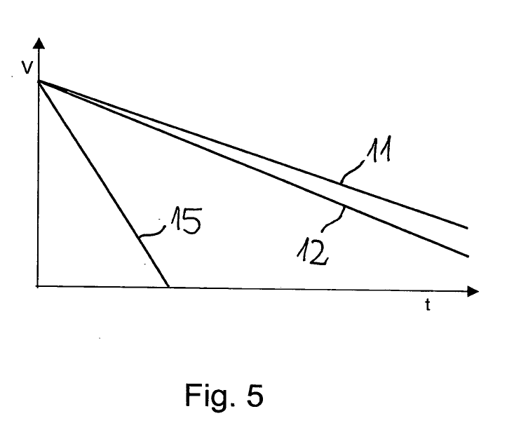

FIG. 5 shows a graphic illustration of various measurement signals, wherein the time t is graphed on the abscissa and the signal amplitude V is graphed on the ordinate,

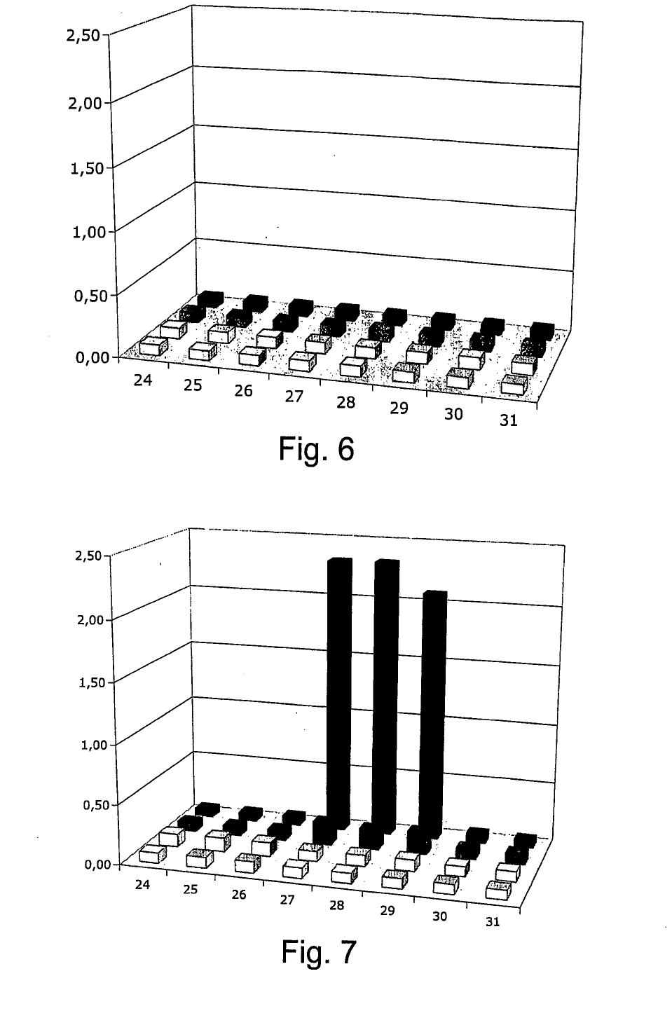

FIG. 6 shows a bar graph of the slopes of the darkness measurement signals of the individual radiation receivers of a semiconductor chip,

FIG. 7 shows a bar graph of the slopes of the radiation measurement signals of the individual radiation receivers of a semiconductor chip,

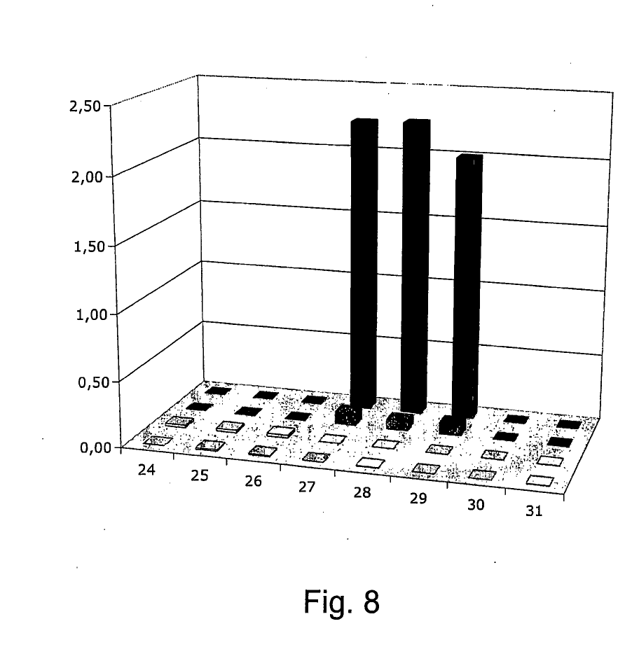

FIG. 8 shows a bar graph of the slopes of the radiation measurement signals relative to the darkness measurement signals for the individual radiation receivers of a semiconductor chip,

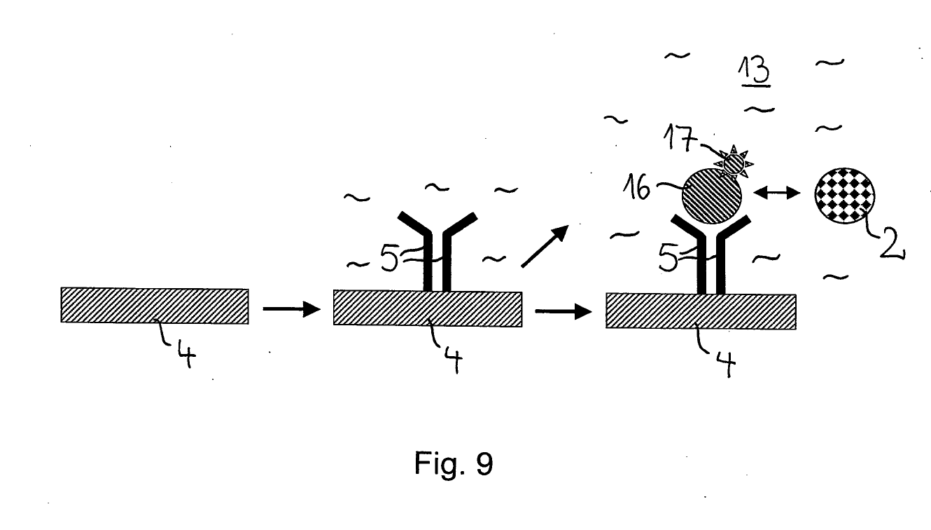

FIG. 9 shows a schematic representation of an indirect ELISA assay system, and

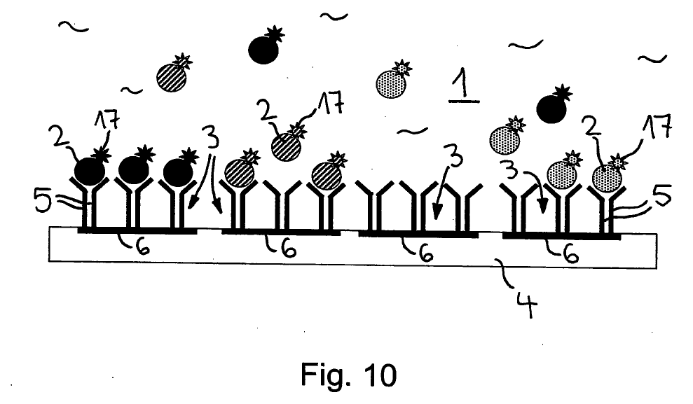

FIG. 10 shows a semiconductor chip on which a plurality of test locations with receptors is arranged, wherein a solution to be analyzed that contains ligands and competitors marked with a luminophore is applied to said semiconductor chip.

In a method for detecting and/or determining the concentration of ligands 2 contained in a solution to be analyzed 1, receptors 5 are immobilized on test locations 3, which are arranged on the surface of a semiconductor chip 4 (FIG. 1). The semiconductor chip 4 has a plurality of test locations 3, which are preferably arranged in matrix form in a plurality of rows and columns and separated from each other.

The receptors 5 are adapted to the ligands 2 in such a way that they enter into a specific bond when they come into contact with said ligands. The immobilization of the receptors 5 can be achieved by applying an adhesive layer to the semiconductor chip 4, on which layer the receptors 5 anchor themselves. Examples of adhesive layers include silane layers and/or polymer layers (compare EP 1 176 423 A1). The receptors 5 can be printed onto the test locations and/or applied in dissolved form to said test locations on said semiconductor chip 4 with a micropipette, which is positioned relative to said semiconductor chip 4 by means of an XY positioning device.

After the receptors 5 have been immobilized on the semiconductor chip 4, the solution to be analyzed is applied to the test locations 3 in order to allow the ligands 2 contained in the solution 1 to bind specifically to the receptors 5 (FIG. 2). Said solution 1 can be applied with the aid of a pipette, which is not shown in any greater detail in the drawing. It is also conceivable, however, that the semiconductor chip 4 is adjacent to a flow-through measurement chamber, which has an inlet opening through which said solution 1 is pumped into said flow-through measurement chamber by means of a micropump.

In FIG. 3, it can be discerned that the solution 1 is then removed, for example, with the aid of a rinse liquid from the test locations 3 so that only those ligands 2 that are specifically bound to a receptor 5 are arranged on said test locations 3. Said rinse liquid can be stored in a reservoir, which is not illustrated in any greater detail in the drawing, and which is capable of being connected to said test locations 3 via a feeder mechanism having a micropump.

In another method step shown in FIG. 3, the ligands 2 remaining on the test locations 3 are brought into contact with biotinylated detection antibodies 7 that bind to said ligands 2, but not, however, to free receptors 5. Receptor-ligand-detection antibody complexes thus form on the locations on which a ligand 2 had bound with a receptor 5 beforehand. Said detection antibodies 7 are stored together with a carrier liquid in another reservoir, which is capable of being connected to said test locations 3 via said feeder mechanism. Obviously, it is also possible to apply the carrier fluid containing the detection antibodies 7 to the test locations 3 with the aid of a pipette.

Any remaining free ligands 2 are now removed from the test locations so that only free receptors, receptor-ligand complexes, and/or receptor-ligand-detection antibody complexes are present on said test locations 3. In order to remove said free ligands 2, said test locations are rinsed with a rinse liquid, which is conducted from a reservoir to said test locations 3 with the aid of said feeder mechanism.

In another method step, the test locations 3 are brought into contact with a tracer solution that contains streptavidin, on which the enzyme horseradish peroxidase 9 is bound. The streptavidin has the property of binding specifically to the biotinylated detection antibody 7. As can be discerned in FIG. 4, the horseradish peroxidase 9 also binds indirectly to the detection antibody 7 by means of the action of the streptavidin. The tracer solution is conveyed from a reservoir to said test locations 3 with the aid of said feeder mechanism.

The surface of the semiconductor chip 4 is now rinsed with a substitute liquid 10, namely a phosphate-buffered saline solution (PBS), in order to remove the tracer solution and any free horseradish peroxidase still contained therein from the test locations 3. Said substitute liquid 10 is stored in a reservoir that is capable of being connected to said test locations 3 via said feeder mechanism.

At this point, the horseradish, peroxidase 9 is only present on the locations on which a ligand 2 is bound to a receptor 5 immobilized on the semiconductor chip 4. The receptor-ligand complexes are thus marked with horseradish peroxidase 9.

It can be discerned in FIGS. 1 through 4 that an electronic radiation receiver 6, for example, a photodiode, is always integrated in the test locations 3 in the semiconductor chip 4. It is clearly discernible that the receptors 5 are always directly immobilized on the radiation receivers 6. Said radiation receivers 6 are sensitive to a luminescence radiation that is generated according to the bond of the ligand 2 to the receptor 5 in a subsequent method step, which will be described in more detail later.

With the aid of the radiation receivers 6, a dark measurement signal 11 is always picked up for the individual test locations 3, while said radiation receivers 6 are in contact with the substitute liquid 10. Said substitute liquid is adapted to the receptors 5, the ligands 2, and the detection antibodies 7 in such a way that no luminescence is generated during the dark measurement.

In FIG. 5 it can be discerned that the radiation receivers 6 are charged to a prespecified electrical voltage at the beginning of the darkness measurement and that the level of the measurement voltage on said radiation receivers 6 continually declines in each case during the darkness measurement. During or after the darkness measurement, a slope value is determined and saved for the darkness measurement signal 11. The corresponding slope values for a semiconductor chip with 32 test locations 3, said test locations being arranged in matrix form in four rows and eight columns, are graphically represented in the form of a bar graph in FIG. 6.

Additionally, a second darkness measurement signal 12, which was recorded with a dry radiation receiver 6, is shown in FIG. 5. It can be clearly discerned that said second darkness measurement signal 12 has a greater slope than the first darkness measurement signal 11. The measurement voltage or the darkness current of said radiation receiver 6 is therefore influenced by the presence of the substitute liquid 10.

After the darkness measurement signals 11 have been measured in each case for the individual radiation receivers 6, the substitute liquid 10 is removed from the test locations 3 and an auxiliary liquid 13, which contains hydrogen peroxide and luminol, is applied to said test locations 3. An example of such an auxiliary liquid is commercially available under the trade name “ECL® Solution.” The ligand is either not present or else present in an insignificant concentration relative to the measurement in said auxiliary liquid 13. Said auxiliary liquid 13 is conveyed from a reservoir to said test locations 3 with the aid of said feeder mechanism.

When the horseradish peroxidase 9 comes into contact with the hydrogen peroxide, free oxygen radicals are split off from the hydrogen peroxide, whereby the luminol is chemically broken down and emits luminescence radiation according to the following equation:

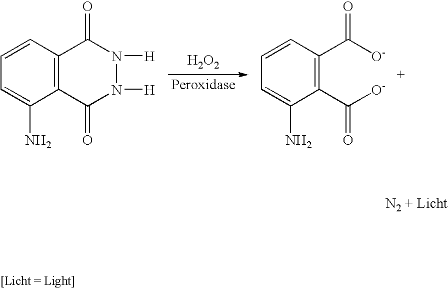

A luminescence radiation 14 is thus generated according to the bond of the ligand 2 to the receptor 5. This radiation has a wavelength of around 428 nm. The luminescence radiation 14 for the individual test locations 3 is detected in each case with the aid of the radiation receiver 6 located on the test location 3, while said receiver is covered with the auxiliary fluid 13. The corresponding radiation measurement signal is designated with 15 in FIG. 5. It can be clearly discerned that said radiation measurement signal 15 is considerably stronger than the darkness measurement signal 11 due to an electrical current induced in said radiation receiver 6 by the luminescence radiation.

The radiation measurement signals 15 of the individual radiation receivers 6 in each case have a more or less linear progression. During or after the pickup of the radiation measurement signal 15, a value for the slope of said radiation measurement signal 15 relative to the darkness measurement signal 11 is determined with the aid of the slope value for the darkness measurement signal 11, said slope value being allocated to the corresponding radiation receiver 6. In FIG. 8, the corresponding relative slope values are graphically represented in each case in the form of a bar graph for the individual radiation receivers 6. The absolute slope values of the radiation measurement signals 15 are given in FIG. 7 for comparison.

The matrix of the semiconductor chip 4 has a plurality of groups of test locations 3, on which the same receptors are always immobilized. Thus, columns 1 through 3 (from left to right) and columns 4 through 6 of each line of the matrix always form a group of test locations 3. Test locations 3 that are allocated to different groups always differ with regard to their receptors. The arithmetic mean is always calculated from the measurement values of test locations 3 that belong to the same group. Measurement tolerances that may be caused by differences in the radiation receivers 6 and/or the occupancy of said radiation receivers 6 by the receptors 5 are compensated for by this means.

It can be clearly discerned in FIG. 8 that, in the first matrix line located in the background of the drawing, a relatively high measurement signal was measured in each case on three measurement points that are allocated to the same group, and that these measurement signals coincide to a large extent. The measurement signal is considerably lower on the corresponding measurement points of the second line. An even lower measurement signal was detected on the first three measurement points of the third line. The measurement signal is around zero on the remaining measurement points.

In the exemplary embodiment shown in FIG. 9, a solution to be analyzed 1 that contains the ligands 2 and competitors 16 is applied to the test location 3 arranged on the semiconductor chip 4. A radiation receiver 6, which is not shown in any greater detail in the drawing, is integrated in the semiconductor chip 4 on the test location 3.

The ligands 2 as well as the competitors 16 always specifically bind to a receptor 5 when they come into contact therewith. In can be discerned in FIG. 9 that said competitors 16 are marked with a luminophore 17. Said luminophore 17 emits a luminescence radiation 14 when it is irradiated with an excitation radiation. The wavelength of said excitation radiation is different from the wavelength of said luminescence radiation 14. The radiation receivers 6 are not sensitive to said excitation radiation.

After the solution to be analyzed 1 has been brought into contact with the test location 3 for a period of time sufficient for a representative quantity of the ligands 2 and/or competitors 16 contained in said solution 1 to bind to the receptors 5, said solution 1 is removed from said test location 3 and replaced with an auxiliary liquid 13 that contains neither ligands 2 nor competitors 16. Afterwards, said test location 3 is irradiated with the excitation radiation in order to stimulate luminophores 17, which are indirectly bound to said receptors 5 via said competitors 16, to emit the luminescence radiation 14. While said test location 3 is being irradiated with said excitation radiation, a radiation measurement signal 15 is picked up with the aid of the radiation receiver 6, which is in contact with said auxiliary liquid 13. Afterwards, said excitation radiation is turned off in order to detect a darkness measurement signal 11 with the aid of said radiation receiver 6, with said auxiliary liquid 13 still in contact therewith.

The radiation measurement signal 15 is compensated for with the darkness measurement signal 11 by subtracting said darkness measurement signal 11 from said radiation measurement signal 15. The slope of said radiation measurement signal 15 relative to said darkness measurement signal 11 is determined with the aid of the compensated measurement signal thus obtained. The compensation can be achieved with the aid of a suitable compensation mechanism, which has inputs for said radiation measurement signal 15 and said darkness measurement signal 11, as well as an output for said compensated radiation measurement signal.

It can be discerned in FIG. 10 that the receptors 5 immobilized on the individual measurement points 3 enter into a specific bond with various ligands. The ligands are always marked with a luminophore 17 that only binds to the ligands, but not, however, to free receptors 5. Said ligands are applied to the test locations 3 in the solution to be analyzed 1. After the solution to be analyzed 1 has been brought into contact with the test location 3 for a period of time sufficient for a representative quantity of the ligands 2 contained in said solution 1 to bind to the receptors 5, said solution 1 is removed from said test location 3 and replaced with an auxiliary liquid 13 that contains no ligands 2 fitting said receptors. Afterwards, said test location 3 is irradiated with excitation radiation in order to stimulate those luminophores 17 that are bound to said receptors 5 via said ligands 2 to emit the luminescence radiation 14. While said test locations 3 are being irradiated with said excitation radiation, the radiation measurement signals 15 are picked up with the radiation receivers 6, which are in contact with said auxiliary liquid 13. Afterwards, said excitation radiation is turned off in order to measure the darkness measurement signals 11 while said radiation receivers 6 remain in contact with said auxiliary liquid 13.

In the method for detecting and/or determining the concentration of a ligand 2 contained in a solution to be analyzed, a receptor 5 that is capable of entering into a specific bond with said ligand 2 is immobilized on a test location 3 on a semiconductor chip 4. The solution is applied to said test location 3 in order for said ligand 2 to bind to said receptor 5. A luminescence radiation 14 or a color change is generated according to the bond of said ligand 2 to said receptor 5. With the aid of a radiation receiver 6 integrated in said semiconductor chip 4, a radiation measurement signal 15 is picked up while the auxiliary liquid 13 is in contact with said radiation receiver 6. A darkness measurement signal 11 is measured for said radiation receiver 6 while the generation of said luminescence radiation 14 is being prevented and while said radiation receiver 6 is in contact with said auxiliary liquid 13 and/or a substitute liquid. Said radiation measurement signal 15 is compensated for with said darkness measurement signal 11.

1. A method for detecting and/or determining the concentration of at least one ligand assumed to be contained in a solution to be analyzed,
a) wherein at least one receptor, which enters into a specific bond with said ligand when said receptor contacts said ligand, is immobilized on at least one test location on a semiconductor chip,
b) wherein said solution is applied to said test location in order to bind said ligand to said receptor on said test location,
c) wherein said solution is removed from said test location and an auxiliary liquid that contains a luminophore that is stimulated to emit a luminescence radiation according to the bond of said ligand to said receptor is applied to said test location,
d) and wherein a radiation measurement signal is picked up with the aid of at least one radiation receiver, which is sensitive to said luminescence radiation and integrated in said semiconductor chip, characterized in that,
e) said radiation measurement signal is picked up while said auxiliary liquid is in contact with said radiation receiver,
f) said radiation receiver is brought into contact with a substitute liquid in which no luminescence radiation is generated,
g) a dark measurement signal is measured for said radiation receiver while said radiation receiver is in contact with said substitute liquid,
h) and said radiation measurement signal is compensated for with said darkness measurement signal.

2. The method as in claim 1, characterized in that the at least one ligand is marked directly and/or via at least one detection antibody bound to said ligand with at least one enzyme, that the luminophore is selected so that it can be stimulated to emit the luminescence radiation in the presence of said enzyme directly or indirectly by the action of a chemical substance contained in the auxiliary solution.

3. The method as in claim 1, characterized in that the at least one enzyme is a peroxidase enzyme, preferably horseradish peroxidase, and that the luminophore is luminol and the chemical substance is hydrogen peroxide.

4. The method as in claim 1, characterized in that the auxiliary solution contains hydrogen peroxide and luminol and the substitute solution preferably contains a phosphate-buffered saline solution (PBS).

5. The method as in claim 1, characterized in that at least one competitor marked with the enzyme is introduced in the solution to be analyzed, and that said solution is then applied to the test location.

6. A method for detecting and/or determining the concentration of at least one ligand presumed to be contained in a solution to be analyzed,
a) wherein at least one receptor, which enters into a specific bond with said ligand when said receptor contacts said ligand, is immobilized on at least one test location on a semiconductor chip,
b) wherein said solution is applied to said test location in order to bind said ligand to said receptor on said test location,
c) wherein at least one luminophore is immobilized on said test location according to the bond of said ligand on said receptor,
d) wherein the test location is irradiated with an excitation radiation that contains at least one wavelength at which said luminophore is stimulated to emit a luminescence radiation,
e) and wherein a radiation measurement signal is picked up with the aid of at least one radiation receiver integrated in said semiconductor chip and sensitive to said luminescence radiation, characterized in that,
f) said radiation measurement signal is picked up while said solution and/or an auxiliary solution not containing the luminophore are/is in contact with said radiation receiver,
g) a darkness measurement signal is measured for said radiation receiver while the generation of said luminescence radiation is being prevented,
h) said radiation receiver is in contact with said solution, said auxiliary liquid, and/or a substitute liquid during the pickup of said darkness measurement signal,
i) and said radiation measurement signal is compensated for with said darkness measurement signal.

7. The method according to claim 6, characterized in that the ligand is marked with the luminophore directly or via at least one detection antibody.

8. The method according to claim 6, characterized in that at least one competitor marked with the luminophore is introduced in the solution to be analyzed, and that said solution is then applied to the test location.

9. A method for detecting and/or determining the concentration of at least one ligand assumed to be contained in a solution to be analyzed,
a) wherein at least one receptor that enters into a specific bond with said ligand when said receptor contacts said ligand is immobilized on at least one test location on a semiconductor chip,
b) wherein said solution is applied to said test location in order to bind said ligand on said receptor,
c) wherein a reagent is brought into contact with said test location and converted according to the bond of said ligand to said receptor via a chemical reaction into an indicator that differs in color from said reagent,
d) wherein the test location is irradiated with an optical radiation,
e) wherein a radiation measurement signal is picked up with the aid of at least one radiation receiver, which is integrated in said semiconductor chip and is sensitive to the color change occurring in the chemical reaction,
f) while said solution and/or an auxiliary liquid containing the reagent is in contact with said radiation receiver,
g) wherein a darkness measurement signal is measured for said radiation receiver while the generation of the optical radiation is being prevented and/or said indicator is being maintained remote from said test location,
h) wherein said radiation receiver is in contact with said solution or said auxiliary liquid and/or a substitute liquid during the pickup of said darkness measurement signal,
i) and wherein said radiation measurement signal is compensated for with said darkness measurement signal.

10. The method as in claim 9, characterized in that the semiconductor chip has a plurality of radiation receivers, and in that a radiation measurement signal and a darkness measurement signal are preferably always picked up for each of said radiation receivers and in that for each of said radiation receivers, the radiation measurement signal allocated thereto is always compensated for with the darkness measurement signal allocated thereto.

11. The method as in claim 10, characterized in that at least one receptor is always immobilized on at least two test locations, that a radiation measurement signal and a darkness measurement signal are always picked up for the radiation receivers allocated to said test locations, and that the individual radiation measurement signals are compensated for with the darkness measurement signal that is always allocated to said individual radiation measurement signals.

12. The method as in claim 11, characterized in that the same receptors are immobilized on the semiconductor chip on at least two test locations to which the solution to be analyzed is applied, in that at least one radiation measurement signal is always picked up and compensated for with a darkness measurement signal for said test locations, and in that an average signal is formed from the compensated measurement signals thus detected.

13. The method as in claim 12, characterized in that the radiation receiver is a photodiode that is connected to an electrical power source in order to be charged to its barrier layer capacity in reverse biasing, in that afterwards the connection to the power supply is interrupted, and that during the interruption the electrical voltage is picked up on said photodiode as a radiation measurement signal and/or a darkness measurement signal.

14. The method as in claim 13, characterized in that the darkness measurement signal is approximated as a line through an intercept, in that the slope of said line is determined, and in that the radiation measurement signal is compensated for with reference to said slope.

15. The method as in claim 14, characterized in that the substitute liquid is a wash solution, in that the at least one test location is rinsed with said wash solution after the application of the solution to be analyzed, in that the darkness measurement signal is then picked up, and in that said wash solution is then replaced with the auxiliary solution and the radiation measurement signal is picked up.

16. A measurement device for detecting and/or determining the concentration of at least one ligand assumed to be contained in a solution to be analyzed, comprising a semiconductor chip on which at least one receptor is immobilized on at least one test location, which receptor enters into a specific bond with said ligand when said receptor contacts said ligand, comprising a first reservoir allocated to an auxiliary liquid containing a luminophore that is capable of being stimulated to emit a luminescence radiation according to the bond of said ligand to said receptor awhile in contact with said test location, comprising at least one radiation receiver, which is sensitive to said luminescence radiation and is integrated in said semiconductor chip, comprising a second reservoir allocated to a substitute liquid not containing the luminophore, comprising a feeder mechanism connecting said reservoirs to said test location, via which mechanism said auxiliary liquid and said substitute liquid can be conveyed separately to said test location, comprising a control and evaluator mechanism, which is connected to said feeder mechanism and said radiation receiver and which is control connected to said feeder mechanism in such a way that said auxiliary liquid and said substitute liquid are sequentially fed to said test location, and wherein the control mechanism is configured so that
it triggers the picking up of a radiation measurement signal while said test location is in contact with said auxiliary fluid, and
it triggers the picking up of a darkness measurement signal while said test location is in contact with said auxiliary fluid,
and so that said control and evaluator mechanism has a compensation mechanism for compensating said radiation measurement signal according to said darkness measurement signal.

17. A measurement device for detecting and/or determining the concentration of at least one ligand assumed to be contained in a solution to be analyzed, comprising a semiconductor chip on which at least one receptor is immobilized on at least one test location, which receptor enters into a specific bond with said ligand when said receptor contacts said ligand, comprising a reservoir allocated to a luminophore capable of being stimulated by irradiation with an excitation radiation to emit a luminescence radiation according to the bond of said ligand to said receptor while in contact with said test location, comprising at least one radiation source to emit said excitation radiation, comprising at least one radiation receiver, which is integrated in said semiconductor chip and which is sensitive to said luminescence radiation, comprising a feeder mechanism connecting said reservoir with said test location and via which mechanism said luminophore is capable of being fed into said test location, comprising a control and evaluator mechanism that is control connected to said feeder mechanism, said radiation receiver, and said radiation source in such a way that a radiation measurement signal is picked up while said radiation source is turned on and said solution is in contact with said radiation receiver, wherein during the picking up of a radiation measurement signal, said radiation source is turned on and during the picking up of a darkness measurement signal, said radiation source is turned off, and wherein said control and evaluator mechanism has a compensation mechanism to compensate for said radiation measurement signal according to said darkness measurement signal.

18. A measurement device for detecting and/or determining the concentration of at least one ligand assumed to be contained in a solution to be analyzed, comprising a semiconductor chip on which at least one receptor is immobilized on at least one test location, which receptor enters into a specific bond with said ligand when said receptor contacts said ligand, comprising a reservoir containing a reagent that is capable of being converted into an indicator while in contact with said test location via a chemical reaction according to the bond of said ligand to said receptor, said indicator differing in color from said reagent, comprising at least one radiation source for irradiating said test location with optical radiation, comprising at least one radiation detector, which is integrated in said semiconductor chip and which is sensitive to the color change occurring in said chemical reaction, comprising a feeder mechanism connecting said reservoir to said test location and via which mechanism said reagent is capable of being fed into said test location, comprising a control and evaluator mechanism that is control connected to said feeder mechanism, said radiation receiver, and said radiation source in such a way that a radiation measurement signal is picked up while said radiation source is turned on and said solution is in contact with said radiation receiver, wherein during the picking up of said radiation measurement signal, said radiation source is turned on and during the picking up of a darkness measurement signal, said radiation source is turned off, and wherein said control and evaluator mechanism has a compensation mechanism to compensate for said radiation measurement signal according to said darkness measurement signal.
